(19) United States
(12) Patent Application Publication
(10) Pub. No.: US 2026/0256584 A1
(43) Pub. Date: Sep. 3, 2026

(54) Annuloplasty device

(71) Applicant: HVR Cardio Oy, Espoo (FI)

(72) Inventor: Olli Keränen, Bjärred (SE)

(21) Appl. No.: 18/872,817

(22) PCT Filed: Jun. 7, 2023
(86) PCT No.: PCT/EP2023/065316

§ 371 Date: Dec. 7, 2024

Related U.S. Application Data
(60) Provisional application No. 63/350,161, filed on Jun. 8, 2022.

(51) Int. Cl. A61F 2/24

(52) U.S. Cl. CPC A61F 2/2445; A61F 2/2466; A61F 2220/0016

(57) ABSTRACT

An annuloplasty device is disclosed comprising a removable and flexible elongate displacement unit insertion into a coronary sinus (CS) adjacent the mitral valve, a proximal reversibly expandable portion being reversibly foldable to an expanded state for positioning against a tissue wall at the entrance of the CS, a distal anchoring portion being movable in relation to the proximal expandable portion in a longitudinal direction of the displacement unit to an activated state in which the shape of the annulus is modified to a modified shape, a stent arranged around the displacement unit and being movable relative the displacement unit along the longitudinal direction for insertion into the CS, and wherein the stent is releasably connected to a delivery device and arranged radially between the displacement unit and the proximal expandable portion in a radial direction.

### FIELD OF THE INVENTION

This invention pertains in general to the field of annuloplasty devices for treating a defective mitral valve. More particularly the invention relates to an annuloplasty device for treating a defective mitral valve via coronary sinus, and a method therefore.

### BACKGROUND OF THE INVENTION

Diseased mitral and tricuspid valves frequently need replacement or repair. The mitral and tricuspid valve leaflets or supporting chordae may degenerate and weaken or the annulus may dilate leading to valve leak. Mitral and tricuspid valve replacement and repair are frequently performed with aid of an annuloplasty ring, used to reduce the diameter of the annulus, or modify the geometry of the annulus in any other way, or aid as a generally supporting structure during the valve replacement or repair procedure.

Implants have previously been introduced into the coronary sinus (CS) in order to affect the shape of the valve annulus and thereby the valve function. WO02/062270 discloses such implant that is aimed to replace annuloplasty rings. Implanting annuloplasty devices in the CS is a procedure that entails several challenges, such as re-shaping the annulus in a manner that sustain proper valve function, and ensuring the correct position of the device in the CS over time. Possible traumatic effects on the CS itself have to be taken into account, as well as the complexity of the implant and the procedure. Prior art devices typically have suboptimal performance in several of the aforementioned aspects of annuloplasty via the CS. A problem is to ensure that a significant part of the annulus is reshaped while providing for atraumatic engagement with the anatomy. A problem with the prior art is complex and difficult-to-operate devices, that may require frequent adjustment and repositioning to ensure the correct function over time. This may have dire consequences for the patient and the health care system. Patient risk is increased.

It is desirable to increase the degree of control of the downsizing procedure, i.e. the re-shaping of the annulus, while ensuring a secure anchoring of the implant and minimal risk of damage to the CS.

Hence, an improved annuloplasty device for performing downsizing and reshaping of the valve annulus would be advantageous and in particular allowing for ensuring long-term functioning, less complex procedure, and less traumatic effects on the anatomy and increased patient safety. Also, a method of downsizing and reshaping the mitral valve annulus with such annuloplasty device would be advantageous.

### SUMMARY OF THE INVENTION

Accordingly, examples of the present invention preferably seek to mitigate, alleviate or eliminate one or more deficiencies, disadvantages or issues in the art, such as the above-identified, singly or in any combination by providing a device according to the appended patent claims.

According to a first aspect an annuloplasty device for treating a defective mitral valve having an annulus is provided, comprising a removable and flexible elongate displacement unit for temporary insertion into a coronary sinus (CS) adjacent the valve, wherein the displacement unit has a delivery state for delivery into the CS, and an activated state to which the displacement unit is temporarily and reversibly transferable from said delivery state, a proximal reversibly expandable portion being reversibly foldable to an expanded state for positioning against a tissue wall at the entrance of the CS, wherein the displacement unit comprises a distal anchoring portion being movable in relation to the proximal expandable portion in a longitudinal direction of the displacement unit to said activated state in which the shape of the annulus is modified to a modified shape when inserted into the CS, a stent arranged around the displacement unit and being movable relative the displacement unit along the longitudinal direction for insertion into the CS, and wherein the stent is releasably connected to a delivery device and arranged radially between the displacement unit and the proximal expandable portion in a radial direction (R), the radial direction (R) being perpendicular to the longitudinal direction.

According to a second aspect a method for treating a defective mitral valve having an annulus is provided, said method comprising: inserting a flexible and removable elongate displacement unit in a delivery state into a coronary sinus (CS) adjacent said valve, positioning a proximal reversibly expandable portion against a tissue wall at the entrance of said CS, anchoring a distal anchoring portion inside the CS, activating the displacement unit in an activated state whereby the distal anchoring portion is moved in a longitudinal direction of the displacement unit to reduce a distance (L) between the distal anchoring portion and the proximal expandable portion such that the shape of the annulus is modified to a modified shape, advancing a stent through the proximal expandable portion and over the displacement unit into the CS, anchoring the stent in the CS to retain the modified shape of the annulus, withdrawing the displacement unit through the stent to remove the displacement unit after temporary activation in the activated state.

Further examples of the invention are defined in the dependent claims, wherein features for the second and subsequent aspects are as for the first aspect mutatis mutandis.

Some examples of the disclosure provide for long-term functioning of a repaired mitral valve.

Some examples of the disclosure provide for less complex downsizing procedures of the mitral valve.

Some examples of the disclosure provide for improved control of the downsizing procedure of the mitral valve.

Some examples of the disclosure provide for a reduced risk of damaging the anatomy such as the CS.

Some examples of the disclosure provide for a secure downsizing while at the same time reducing the risk of damaging the anatomy such as the CS.

Some examples of the disclosure provide for improved downsizing of the mitral valve annulus while ensuring an atraumatic procedure.

Some examples of the disclosure provide for reduced risk of long-term negative effects of CS implants.

It should be emphasized that the term “comprises/comprising” when used in this specification is taken to specify the presence of stated features, integers, steps or components but does not preclude the presence or addition of one or more other features, integers, steps, components or groups thereof.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a schematic illustration of an annuloplasty device, in a cross-sectional view, according to one example;

FIG. 2a is a schematic illustration of an annuloplasty device, according to one example;

FIG. 2b is a schematic illustration of the annuloplasty device in FIG. 2a, with an expanded proximal portion, according to one example;

FIGS. 3a-b are schematic illustrations of an annuloplasty device, with different lengths between the proximal expandable portion and the distal anchoring portion, according to one example;

FIG. 4a is a schematic illustration of an annuloplasty device, where a catheter is advanced over an elongated displacement unit, according to one example;

FIG. 4b is a schematic illustration of the annuloplasty device in FIG. 4a, where a stent is advanced over the elongated displacement unit, according to one example;

FIG. 4c is a schematic illustration of the annuloplasty device in FIG. 4b, where the stent is exposed in position over the elongated displacement unit, according to one example;

FIG. 4d is a schematic illustration of the annuloplasty device in FIG. 4c, where the elongated displacement unit is withdrawn through the stent, according to one example;

FIG. 4e is a schematic illustration of the annuloplasty device in FIG. 4d, in a cross-sectional view across the radial direction, according to one example;

FIGS. 5a-b are schematic illustrations of an annuloplasty device positioned in the coronary sinus (CS), where an elongated displacement unit has been anchored with proximal expandable portion and a distal anchor, according to one example;

FIGS. 5c-d are schematic illustrations of an annuloplasty device, where a catheter has been advanced through the proximal expandable portion and over the elongated displacement unit, according to one example;

FIGS. 5e-f are schematic illustrations of an annuloplasty device, where a stent has been advanced in the catheter of FIGS. 5c-d, according to one example;

FIGS. 5g-h are schematic illustrations of an annuloplasty device, where the stent of FIGS. 5e-f has been at least partly expanded in the CS, according to one example;

FIG. 5i is a schematic illustration of an annuloplasty device, where the stent of FIGS. 5e-f has been fully expanded in the CS, according to one example;

FIG. 5j is a schematic illustration of an annuloplasty device, where the elongated displacement unit of FIG. 5i has been withdrawn through the stent, according to one example;

FIGS. 5k-l are schematic illustrations of an annuloplasty device, where the stent of FIGS. 5e-f has been fully expanded and implanted in the CS, according to one example;

FIG. 6a is a schematic illustration of a mitral valve and the adjacent coronary sinus;

FIG. 6b is a schematic illustration of an annuloplasty device, where the stent of FIGS. 5e-f has been fully expanded and implanted in the CS to re-shape the annulus, according to one example;

FIG. 7 is a schematic illustration of an annuloplasty device, in a cross-sectional view, according to one example;

FIG. 8a is a flow chart of a method for treating a defective mitral valve according to one example;

FIG. 8B is a flow chart of a method for treating a defective mitral valve according to one example;

FIGS. 9a-c are schematic illustrations of a section of a stent of the annuloplasty device, where the stent has a plurality of retention units, according to examples of the disclosure;

FIG. 10 is a schematic illustration of a section of a stent of the annuloplasty device, where the stent has a plurality of retention units, according to an example;

FIGS. 11a-d are schematic illustrations of a retention units of a stent of the annuloplasty device, according to examples of the disclosure;

FIGS. 12a-b are schematic illustrations of a stent of the annuloplasty device, in a perspective view (12a) and in a side view (12b), where the stent has a plurality of retention units, according to examples of the disclosure;

FIGS. 13a-b are schematic illustrations of a stent of the annuloplasty device, in a perspective view (13a) and in a side view (13b), where the stent has a plurality of retention units, according to examples of the disclosure; and

FIG. 14 is a schematic illustration of a stent of the annuloplasty device in a cross-sectional view, according to an example.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

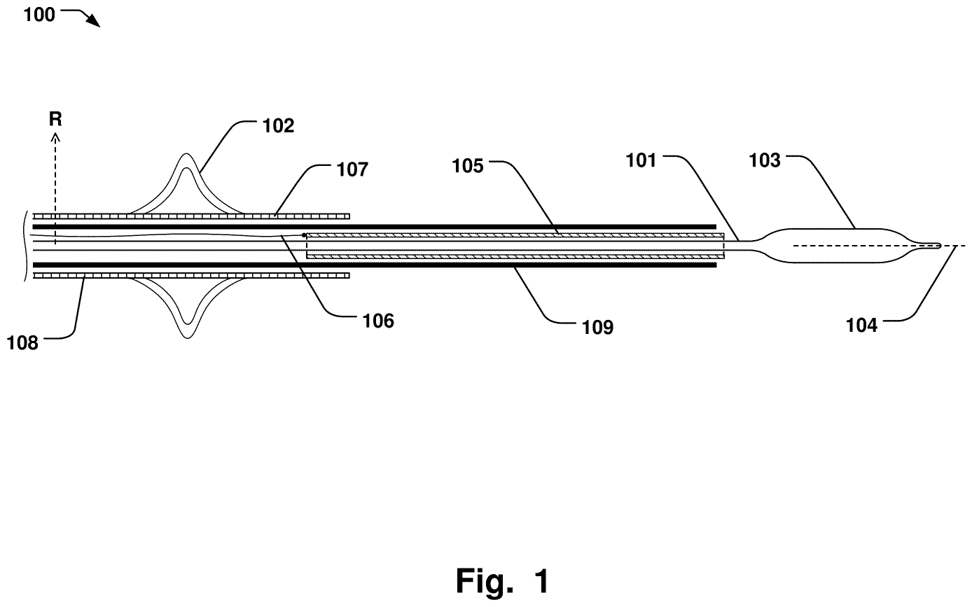

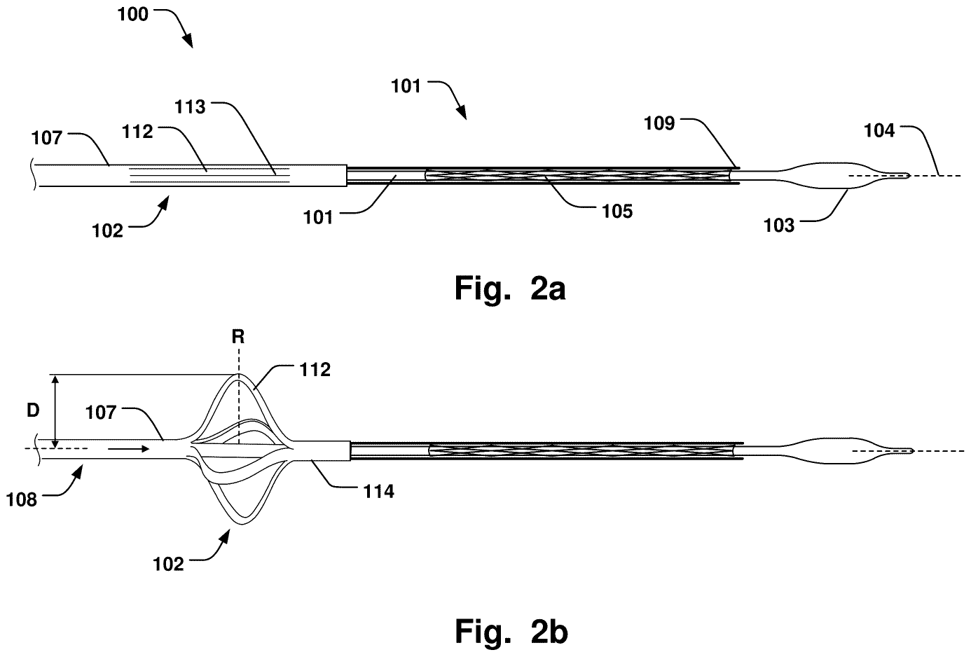

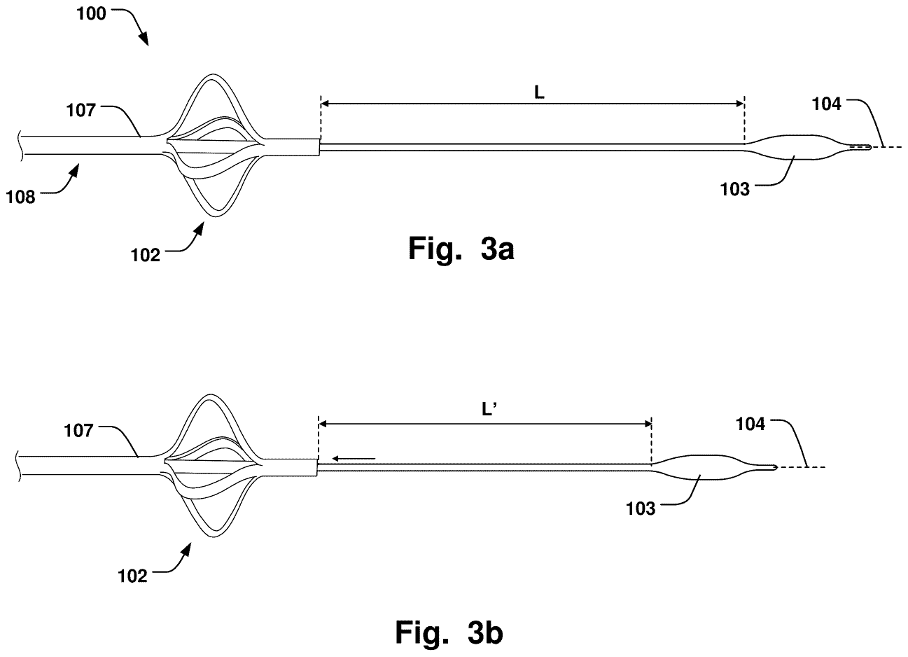

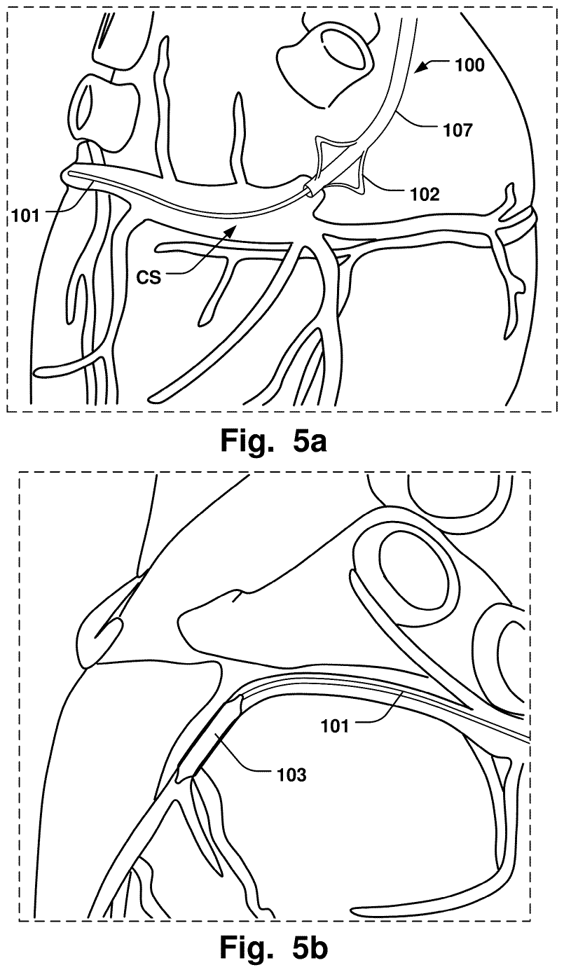

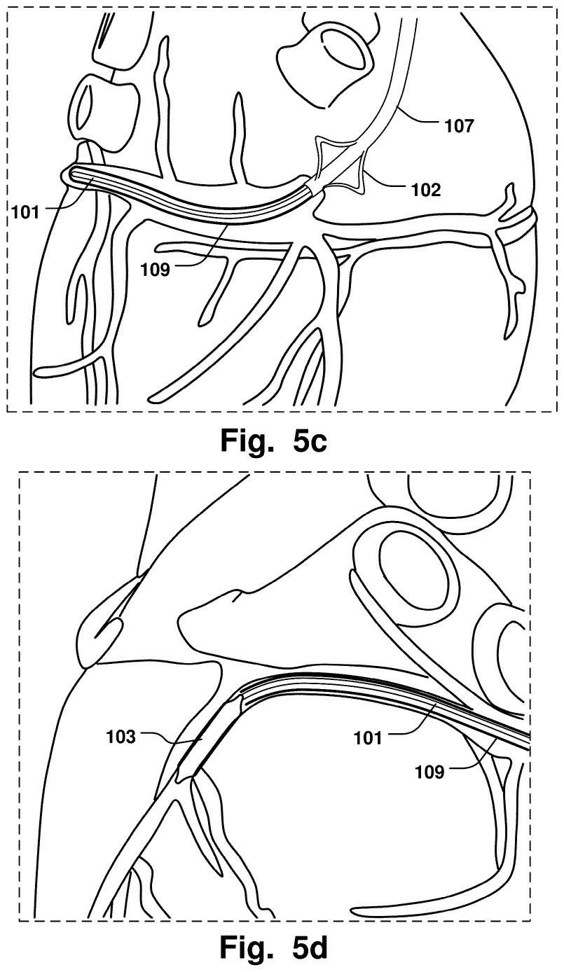

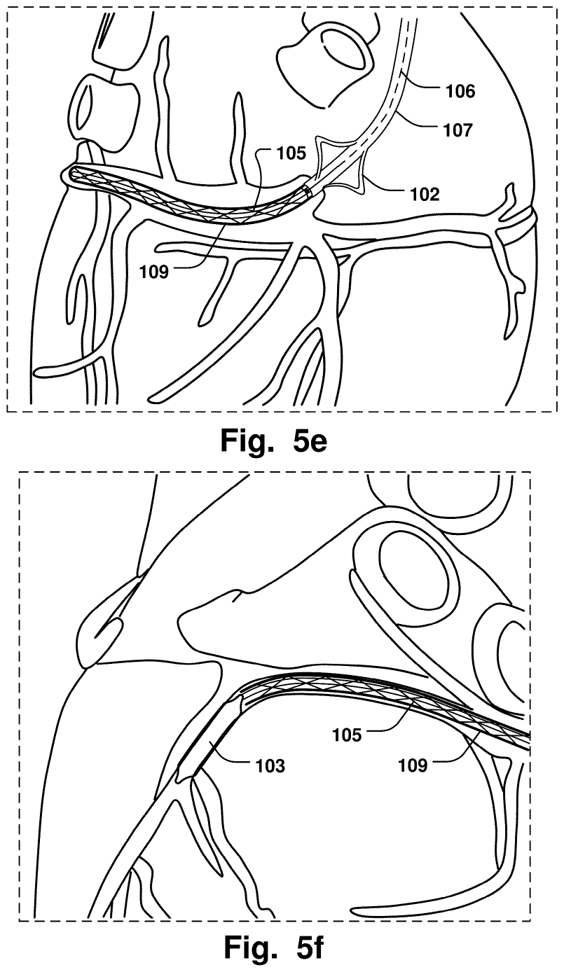

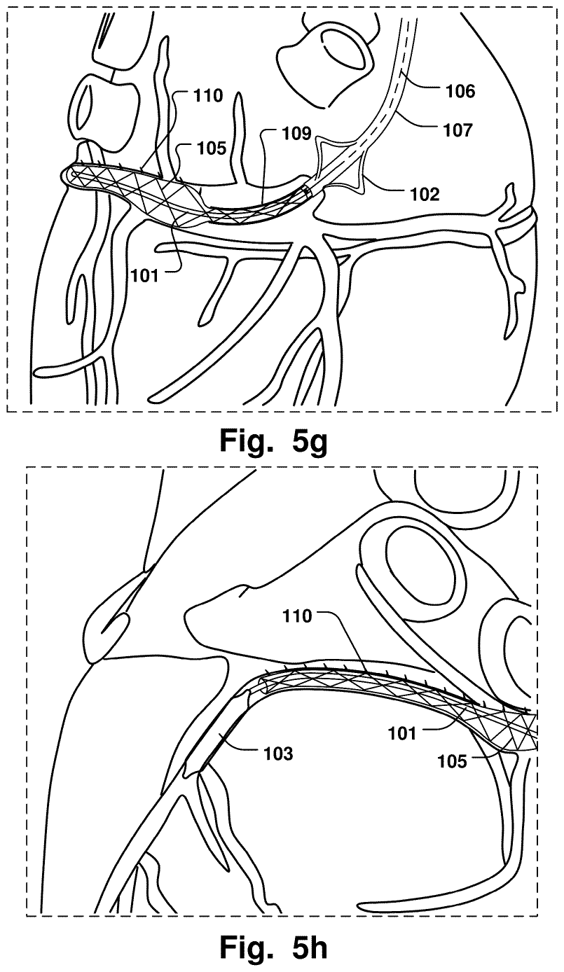

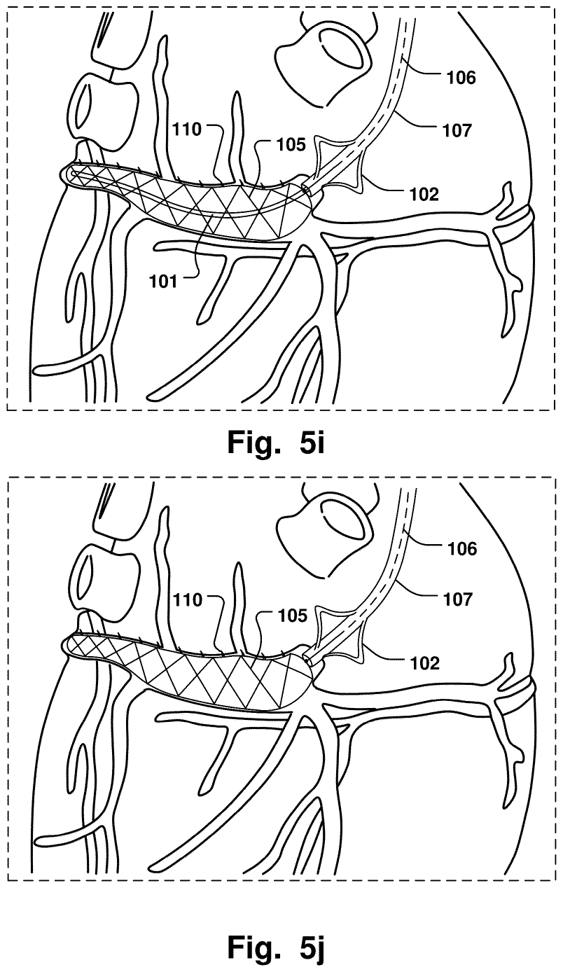

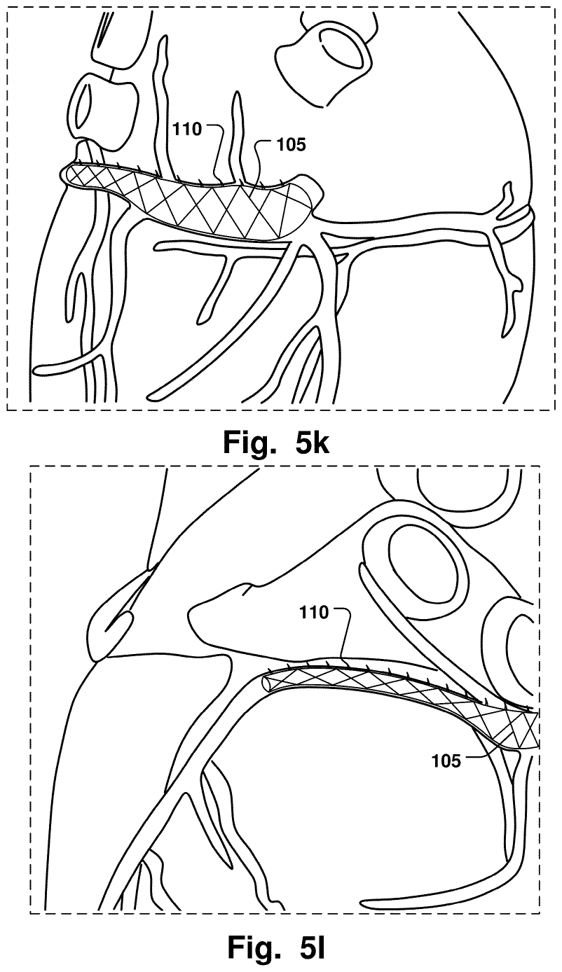

FIG. 1 schematically illustrates an annuloplasty device 100 for treating a defective mitral valve having an annulus. The annuloplasty device 100 comprises a removable and flexible elongate displacement unit 101 for temporary insertion into a coronary sinus (CS) adjacent the mitral valve. The displacement unit 101 has a delivery state for delivery into the CS, and an activated state to which the displacement unit 101 is temporarily and reversibly transferable from said delivery state. The annuloplasty device 100 comprises a proximal reversibly expandable portion 102. The proximal expandable portion 102 is reversibly foldable to an expanded state for positioning against a tissue wall at the entrance of the CS. FIGS. 2a-b show an example where the proximal expandable portion 102 moves from a collapsed state in FIG. 2a, to an expanded state in FIG. 2b. FIG. 5a illustrates schematically how the proximal expandable portion 102 is positioned outside the CS, for pushing against the wall at the entrance of the CS. The displacement unit 101 comprises a distal anchoring portion 103 being movable in relation to the proximal expandable portion 102 in a longitudinal direction 104 of the displacement unit 101 to the aforementioned activated state. FIG. 5b illustrates schematically how the distal anchoring portion 103 is anchored in the CS. The distal anchoring portion 103 may be anchored in the great cardiac vein. FIGS. 3a-b illustrate an example where the distal anchoring portion 103 is movable such that a distance between the proximal expandable portion 102 and the distal anchoring portion 103 is varied. The distance is reduced from a length denoted L in FIG. 3a to a reduced length denoted L′ in FIG. 3b. In the activated state, the annuloplasty device 100, when placed in the CS, modifies the annulus to a modified shape where the annulus is downsized and the leaflets may co-apt. Thus, when the annuloplasty device 100 is placed in the CS as exemplified in FIGS. 5a-b, and the distal anchoring portion 103 is anchored, the latter may be withdrawn towards the proximal expandable portion 102 which exerts a counter force against the tissue wall at the entrance of the CS. This allows for re-shaping the annulus of the mitral valve.

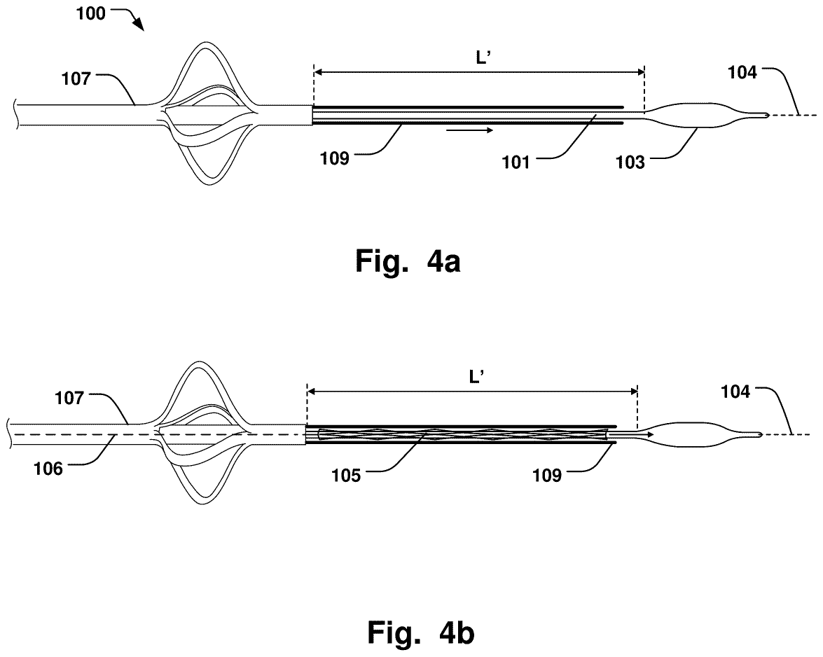

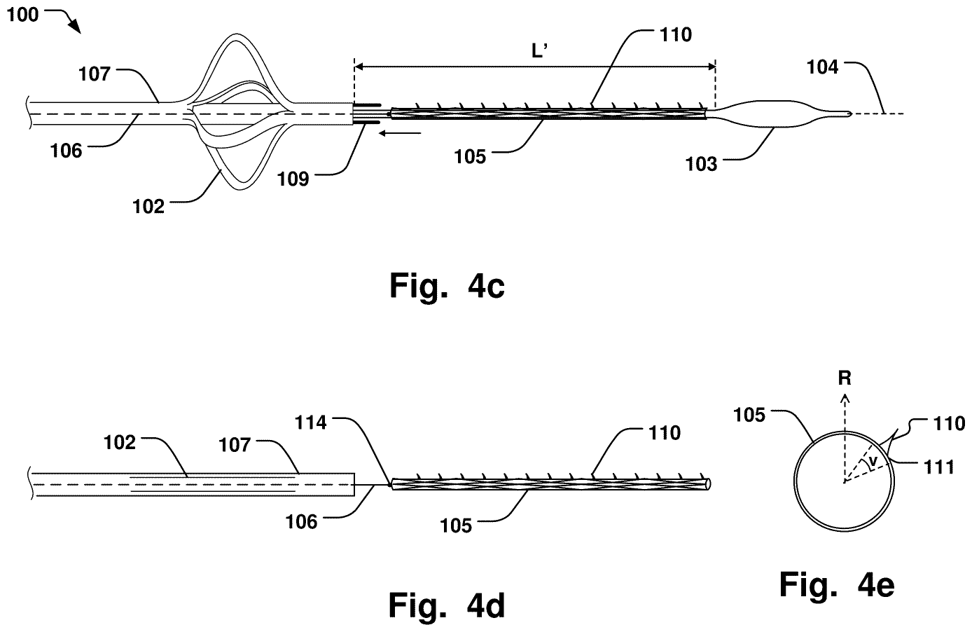

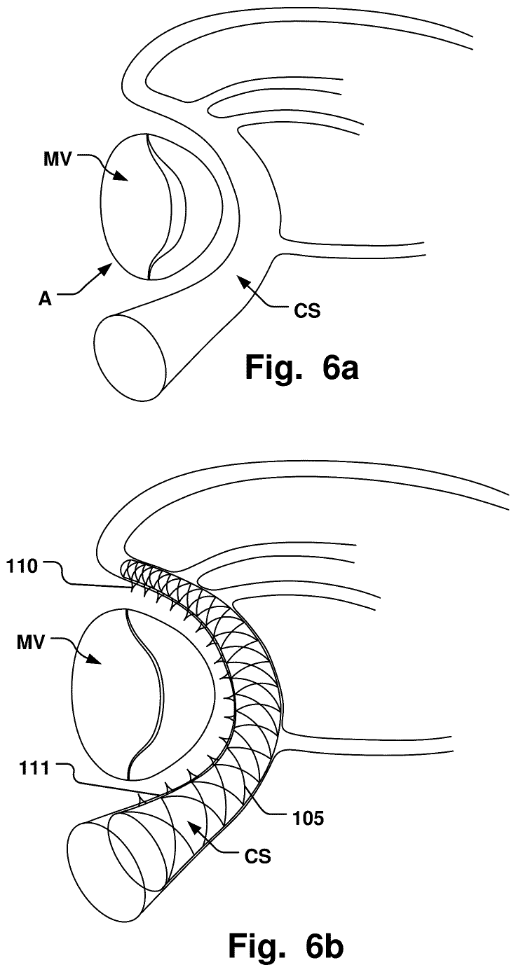

Turning again to FIG. 1, the annuloplasty device 100 comprises a stent 105 arranged around the displacement unit 101 and being movable relative the displacement unit 101 along the longitudinal direction 104 for insertion into the CS. FIG. 4b illustrate schematically how the stent 105 is advanced over the displacement unit 101 towards the distal anchoring portion 103. FIGS. 5e-f illustrate the position of the stent 105 over the displacement unit 101 when the annuloplasty device 100 is placed in the CS. The stent 105 may be expanded for anchoring into the CS when the displacement unit 101 has re-shaped the annulus, as illustrated in FIGS. 5g-i and described further below. The stent 105 is releasably connected to a delivery device 106 as schematically indicated in FIGS. 4d and 5j, and may be released in the CS to maintain the re-shaped form of the annulus after the displacement unit 101 has been withdrawn, as illustrated in FIGS. 5j-l, and 6b. FIG. 6a is an illustration of the heart showing the CS in relation to the mitral valve (MV) in a top-down view. The CS lies adjacent the MV and follows a curvature around the annulus (A) of the MV. The stent 105 may have a releasable connection 114 to the delivery device 106 as exemplified in FIG. 4d. It is conceivable that the delivery device 106 may be configured push or pull the stent 105 relative the displacement unit 101 along the longitudinal direction 104. The stent 105 is arranged radially between the displacement unit 101 and the proximal expandable portion 102 in a radial direction (R), as illustrated in e.g. FIG. 1. The radial direction (R) is perpendicular to the longitudinal direction 104. Having the stent 105 arranged between the displacement unit 101 and the proximal expandable portion 102 allows for advancing the stent 105 over the displacement unit 101 into the CS while the proximal expandable portion 102 is expanded at the outside of the CS and the displacement unit 101 is in the activated state. The re-shaping of the annulus may thus be carefully controlled and optimized with the displacement unit 101, by varying the length (L), before the stent 105 is positioned and finally anchored in the CS. Once the leaflets co-apt properly and no regurgitation occurs, e.g. by observing flow characteristics and leaflet movement, the stent 105 may be expanded gradually (FIGS. 5g-i), e.g. by withdrawing a catheter 109 arranged over the stent 105, as described further below. Connection to the stent 105 can be maintained with the delivery device 106 after the stent 105 has been fully expanded (FIG. 5i). The force exerted by the displacement unit 101 on the annulus may then be gradually released, e.g. by reducing the tension between the proximal expandable portion 102 and the distal anchoring portion 103. The flow characteristics and leaflet movement may be continuously observed to ensure no regurgitation occurs. The displacement unit 101 can be fully withdrawn through the stent 105 (FIG. 5j), and the stent 105 can be released (FIGS. 5k-l) if no regurgitation occurs. Otherwise the stent 105 may be captured, e.g. by advancing a catheter 109 overt the stent 105, and the re-shaping of the annulus may be adjusted further with the displacement unit 101, and/or another stent 105 of a different size may be introduced through the proximal expandable portion 102 and over the displacement unit 101 to repeat the procedure.

It is conceivable that the stent 105 may be advanced through the proximal expandable portion 102 and over the displacement unit 101 into the CS (FIGS. 5e-f), before or after the displacement unit 101 has re-shaped the annulus. Regardless, the stent 105 is expanded and fixed in the CS after the re-shaping has been performed.

The annuloplasty device 100 thus provides for a facilitated annuloplasty procedure via the CS. The re-shaping of the annulus can be carefully controlled and optimized with the displacement unit 101 and the stent 105 can be anchored in the CS to maintain the modified shape when the proper valve function can be confirmed. The safety of the procedure is improved as the position of the stent 105 relative the displacement unit 101 and the CS can be varied and optimized while the displacement unit 101 already provides the downsizing effect of the valve in the activated state. The need to introduce complex elements into the implanted device, i.e. the stent, in order to provide downsizing of the valve can be dispensed with due to the above described cooperation between the displacement unit 101 and the stent 105. The stent 105 may thus be more robust and less complex, and therefore more reliable in sustaining the desired function of the valve over time. Prior art implants may on the contrary require reoccurring adjustments, due to a complex interplay between several moving parts in order to provide downsizing of the annulus. The annuloplasty device 100 provides also for reducing the risk of damaging the CS as the downsizing may be provided by an atraumatically shaped displacement unit 101, instead of the stent 105 which may have retention units 110 as described further below. The risk of tearing of the tissue in the CS with such retention units may thus be reduced.

As described, the distance (L) between the proximal expandable portion 102 and the distal anchoring portion 103 in the longitudinal direction 104 may decrease to a reduced distance (L') when the displacement unit 101 is transferred from the delivery state to the activated state. The proximal expandable portion 102 and the distal anchoring portion 103 may be connected to different sheaths or wires, that may be independently movable in the longitudinal direction 104 to provide for varying the distance (L) as illustrated in FIGS. 3a-b.

The proximal expandable portion 102 may be connected to a sheath 107 and may be configured to be expanded in a radial direction (R), perpendicular to the longitudinal direction 104, by pushing a proximal portion 108 of the sheath 107 towards the distal anchoring portion 103, as indicated in FIG. 2b (see arrow adjacent sheath 107). This provides for a facilitated deployment of the expandable portion 102 to the expanded configuration.

As the displacement unit 101 is positioned with a separation (L′) between the proximal expandable portion 102 and the distal anchoring portion 103 that provides the desired downsizing of the valve, the stent 105 is movable into position over the displacement unit 101 for positioning and anchoring into the CS. The annuloplasty device 100 may comprise a catheter 109 to enclose the stent 105 and position the stent 105 relative the displacement unit 101 in the longitudinal direction 104, as schematically illustrated in FIGS. 4a-b, and FIGS. 5c-f. FIG. 4a show an example where the catheter 109 is first advanced over the displacement unit 101, before the stent 105 is pushed forward inside the catheter 109 by a delivery device 106. It is conceivable however that the stent 105 may be advanced over the displacement unit 101 at the same time as the catheter 109. The stent 105 may be ejectable from, and retrievable into, the catheter 109 by the aforementioned delivery device 106. FIG. 4c is a schematic illustration where the catheter 109 has been withdrawn to expose the stent 105 over the displacement unit 101. FIG. 4d is a schematic illustration where the displacement unit 101 has been withdrawn through the stent 105. The proximal expandable portion 102 is collapsed and the stent 105 may be released from the delivery device 106.

The catheter 109 may thus be movable inside the sheath 107 in the longitudinal direction 104. The stent 105 may thus be positioned at the desired position over the displacement unit 101 while the proximal expandable portion 102, being connected to the sheath 107, is expanded and anchored against the entrance of the CS.

The catheter 109 may thus be movable over the displacement unit 101, and inside said sheath 107, in the longitudinal direction 104, in the aforementioned activated state. This provides for an efficient and reliable positioning and deployment of the stent 105 in the CS while the amount of downsizing of the annulus is effectively controlled by the displacement unit 101.

The stent 105 may be reversibly expandable in the radial direction (R) in the activated state. Thus, once expanded, e.g. as illustrated in the (partly) expanded state in FIG. 5g, the stent 105 may be collapsed and retrieved again if needed in order to re-position or replacing the stent 105. For retrieval, the stent 105 may be pulled into the catheter 109 by withdrawing delivery device 106 relative the catheter 109, thereby forcing the stent 105 into the confinement of the catheter 109. The stent 105 may be self-expandable such that it strives towards an expanded diameter once being release from the catheter 109. The stent 105 may in such case be formed from a shape memory material which has been heat set in an expanded diameter configuration where the diameter is larger than the diameter of the CS. The stent 105 may be compressed and inserted into the catheter 105 before being ejected in the CS where the stent 105 will strive towards the heat set shape and thus press against the tissue walls inside the CS. It is also conceivable that the stent 105 may be actively expanded by e.g. a balloon catheter pushing inside the stent 105 to expand its diameter.

The stent 105 may comprise retention units 110 to anchor the stent 105 in the CS, as schematically illustrated in FIGS. 4c-d, 5g-l, and 6b. Thus, once the displacement unit 101 has contracted the tissue around the CS to re-shape the annulus, by shortening from a length (L) to a reduced length (L′) (FIGS. 3a-b), the shape of the remodelled tissue may be retained by the stent 105 which is anchored into the tissue. Having retention units 110 provides for an effective and reliable anchoring of the stent 105 into the tissue. The stent 105 may extend with an uninterrupted length in abutment with the tissue wall of the CS, along the majority of the length of the CS. This provides for a reliable retention of the re-shaped annulus over time, in particular when having retention units 110 extending essentially along the full length of the stent 105. The stent 105 may have a length that corresponds essentially to the length of the CS, as schematically indicated in FIGS. 5k-l. Retention units 110 may be provided along the length of the stent 105. This further provides for particularly reliable retention of the re-shaped annulus over time.

The retention units 110 may be arranged on a surface section 111 of the stent 105 being adapted to be arranged towards the annulus when the stent 105 is in the CS, as schematically indicated in the top-down view of FIG. 6b in conjunction with FIG. 4e showing a cross-section of the stent 105 in one example. The retention units 110 may thus be arranged at a determined circle sector (v) on the surface of the stent as indicated in FIG. 4e. Having the retention units 110 arranged in a direction towards the annulus provides for an effective retention of the tissue along the corresponding segment of the CS and a reliable retention of the modified annulus shape. The stent 105 may comprise at least one radiopaque marker (not shown) in one example. This provides for a facilitated orienting of the stent 105 in relation to the direction of the annulus.

It is conceivable that a plurality of retention units 110 may be arranged around the circumference of the stent 105 in one example. Retention units 110 may thus be arranged at a plurality of circle sectors (v) along the circumference of the stent 105. This may be advantageous in some applications where an increased retention force is desirable.

The retention units 110 may be shaped to pierce into tissue in the CS and thereby provide a retention force into the tissue. The retention units 110 may be formed from the material of the stent 105. The retention units 110 may thus be integrated with the stent 105. The retention units 110 may thus be cut as respective elongated structures with piercing tips within the structural framework of the stent 105. Forming the retention units 110 as integrated structures of the framework of the stent 105 provides for robust and strong retention units 110 and a minimized risk of dislocations or deformations thereof over time. An overall robust and reliable fixation mechanism is thus provided. The retention units 110 may be formed by different cutting techniques such as by laser cutting techniques.

The retention units 110 may be resiliently moveable from a retracted state to an expanded state. Thus, the retention units 110 may be flexible to bend from the expanded state to the retracted state when arranged inside the catheter 109, and to expand from the retracted state to the expanded state when released from the catheter 109. This provides for a facilitated delivery of the stent 105 through the catheter 109, while allowing for expansion and anchoring of the retention units 110 into the tissue once deployed from the confinement of the catheter 109. The retention units 110 may thus be heat-set to assume a defined expanded shape, as indicated in e.g. FIGS. 4d-e. The expanded shape may thus correspond to a relaxed state of the retention unit 110 where the latter is not acted upon by external forces. The retention unit 110 may thus have a bias towards the expanded shape, by striving towards the relaxed expanded state, when released from the catheter 109.

The retention units 110 may be are aligned essentially flush with an outer diameter of the stent in the retracted state. This provides further for a facilitated delivery of the stent 105 through the catheter 109, as friction between the retention units 110 and the inside lumen of the catheter 109 may be reduced. Further, a compact cross-section is provided and a minimized risk of abrasion and damage to the catheter 109.

In one example the retention units 110 may comprise a shape-memory material, where activation of the shape-memory material causes the retention units 110 to transfer from the retracted state to the expanded state. For example, the shape-memory material may be temperature activated, so that the retention units 110 move towards the expanded state when subject to heating to the body temperature. This provides for an advantageous deployment of the retention units 110 in some applications.

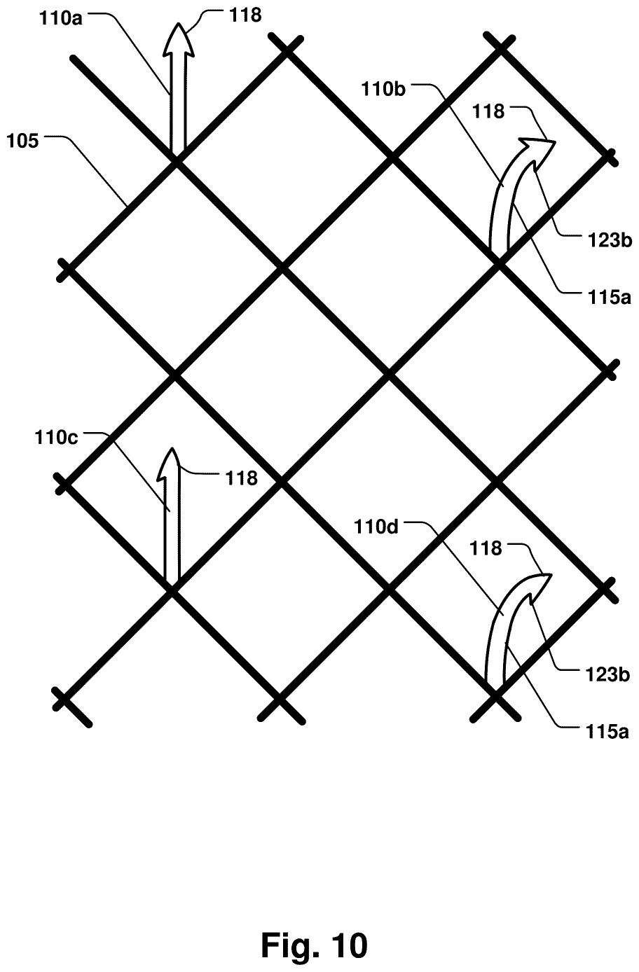

The retention units 110 may be curved in some examples. FIGS. 9-10, 12-13 are schematic illustrations of retention units 110a-d having curved shapes. The stent 105 as described above may have curved retention units 110a-d. Retention units 110 as described above may be retention units 110a-d as described in relation to FIGS. 9-13.

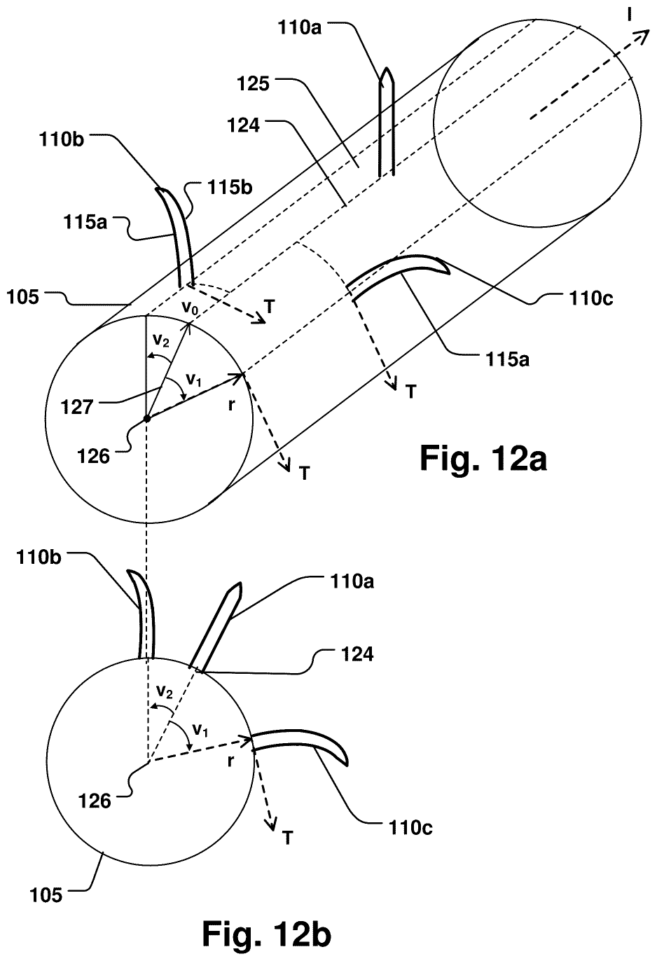

The retention units 110a-d may comprise an at least partly concave surface 115a, or an at least partly convex surface 115b, towards the direction of a respective tangent vector (T) at the respective positions of the retention units 110a-d on the stent 105. The tangent vector (T) is perpendicular to a radial direction (r) of the stent 105 as well as to a longitudinal direction (I) of the stent 105. The radial direction (r) of the stent 105 may be parallel with the above discussed radial direction denoted (R). The longitudinal direction (I) of the stent 105 may be parallel with the above discussed longitudinal direction denoted (L). The radial direction (r) is perpendicular to the longitudinal direction (I). FIG. 12a shows an example of the stent 105 in a perspective view where a retention unit 110c is curved by having a concave shape towards the tangent vector (T) at the radial position of the retention unit 110c on the circumferential surface 125 of the stent 105. The radial position is in this example indicated by the angle v₁ relative a longitudinal reference line 124 on the surface 125. The surface 125 may be construed as a tubular shape with a radius corresponding to the radius of the stent framework, as schematically indicated by the retention unit 110c extending from the surface 125. The longitudinal reference line 124 may be defined as a reference position on the stent 105, and may be positioned in a direction 127 from a center 126 of the stent 105, where the direction 127 represents a reference “zero” angle Vo from the center 126, as indicated in the example of FIG. 12a. The direction 127 may be defined to be aligned with other reference directions, for example aligned in parallel with the axis denoted R (FIG. 4e).

FIGS. 12a-b shows an example of a retention unit 110b being curved with a convex surface 115b facing the direction of the associated tangent vector (T) on the surface 125 where the retention unit 110b is positioned, indicated at radial position v₂, relative the “zero” angle v₀. The tangent vectors (T) of the respective retention units 110a-d, are oriented in the same clockwise direction throughout the examples, provided the circumferential cross-section of the stent 105 (FIG. 12b) as viewed from the indicated center point 126 and along the longitudinal direction (I) in FIG. 12a. FIG. 9a shows a section of the stent 105 and a corresponding alignment of retention units 110b and 110c as exemplified in FIGS. 12a-b. I.e. the retention units 110b, 110c, have convex and concave surfaces 115b, 115a, respectively, towards the tangent vector (T). The example in FIGS. 9a and 12a shows retention unit 110a extending essentially straight so that there is no curvature in the direction of the tangent vector (T). It should be understood that the stent 105 may comprise any combination of retention units 110a-d having different curvatures or extending straight. Further examples are described with reference to FIGS. 9a-c, 10, 12-13.

Having curved retention units 110a-d as described above provides for an improved grip of the stent 105 into the tissue, and thereby a more reliable anchoring of the stent 105 in the CS. The stent 105 may be subject to the repeated movement pattern of the beating heart, when placed in the CS. Curved retention units 110a-d provide for an advantageous directionality, as well as variability, of the retention force by which the respective unit 110a-d is anchored into the tissue. The risk for migration and dislocation over time of the stent 105, onto which the retention units 110a-d are arranged, can thus be reduced. Having retention units 110a-d which are curved in different directions as exemplified above with reference to FIGS. 9a and 12a-b provides for an advantageous anchoring into the tissue of the CS with such reduced risk of migration of the stent 105. For example, the curvature of retention unit 110c in FIG. 12b generates a retention force with a force component aligned in the direction of v₁ towards the center 126, while curved retention unit 110b generates a retention force with a force component aligned in the direction of v₂ towards the center 126. The two aforementioned force components thus intersect with an angle (v₁+v₂) at the center 126 which provides for a secure fixation of the position of the stent 105.

The retention units 110a-d may be curved in opposite directions so that a concave surface 115a of at least a first retention unit 110b faces the opposite direction of a concave surface 115a of at least a second retention unit 110c. FIG. 9a shows an example of a first retention unit 110b being curved opposite a second retention unit 110c. The associated tangent vectors (T) are directed in the same clockwise direction, as described above in relation to FIG. 12a. The concave surface 115a of the first retention unit 110b face away from the direction of the tangent vector (T), while the concave surface 115a of the second retention unit 110c face the same direction as the tangent vector (T). FIG. 9c shows another example where retention units 110c and 110d are curved in opposite directions. The stent 105 may have retention units 110a-d being curved in any combination of directions. Having retention units 110a-d curved in opposite directions provides further for efficiently diverting the retention force with a greater angular spread, and thereby for reducing the risk of dislocation of the stent 105 and for an efficient anchoring into the tissue.

The retention units 110a-d may be curve towards eachother. In the example of FIG. 9c, the first retention unit 110c is curved towards the second retention unit 110d. In another example the first retention unit 110c and the second retention unit 110d are curved away from eachother, as schematically illustrated in FIG. 9b. The stent 105 may have retention units 110a-d being curved in away from eachother, or towards eachother, in any combination.

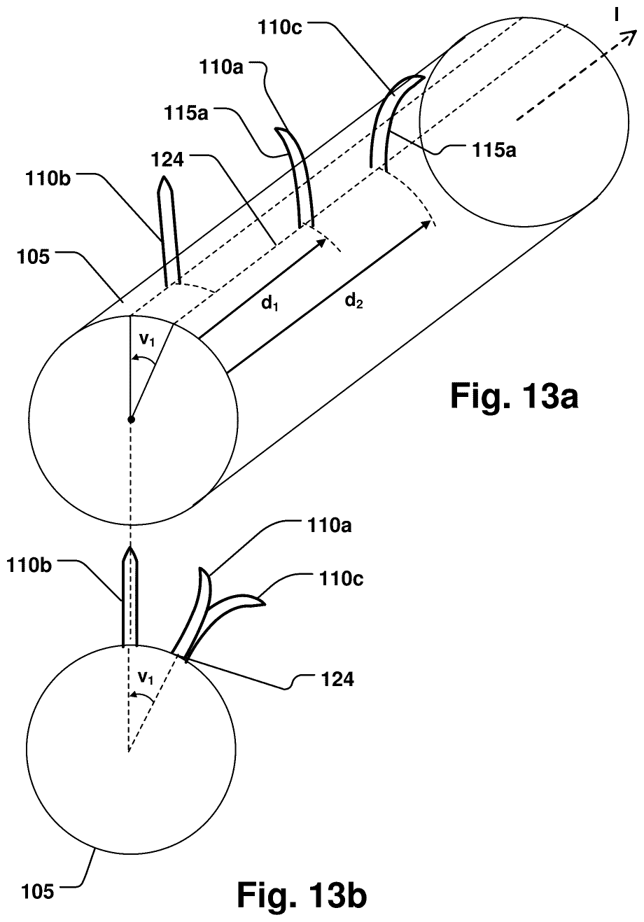

The amount of curvature of the curved shape may vary with an angle (v₁, v₂) at which the respective retention units 110a-d are positioned around a circumference of the stent 105. FIGS. 13a-b show an example where retention unit 110c arranged at angle v₁, relative v₀, has a curved surface 115a which is different from the curved surface 115a of retention unit 110b arranged at v₂. The radius of curvature of the aforementioned curved surfaces 115a may be different. E.g. the radius of curvature of retention unit 110c may be greater than the radius of curvature of retention unit 110b. The example in FIG. 9b shows a further retention unit 110a having the least radius of curvature, e.g. an essentially straight extension. FIGS. 9a and 9c show further examples where the radius of curvature of the retention units 110a-d varies with their respective positions around the circumference of the stent 105. This provides for optimizing the retention force of the retention units 110a-d in dependence on which part on the stent 105 the retention units 110a-d are placed. The stent 105 has a certain extension in the CS where the surrounding anatomy varies along the different parts of the stent 105. The placement and curvatures of the retention units 110a-d may thus be optimized to such varying anatomy to further increase the reliability of the anchoring.

The amount of curvature of the curved shape may vary with a position (d₁, d₂) along the longitudinal direction (I) of the stent 105 at which the respective retention unit 110a-d is positioned. FIGS. 13a-b show an example where retention unit 110a arranged at position d₁, along longitudinal direction (I), has a curved surface 115a which is different from the curved surface 115a of retention unit 110c arranged at a different position d₂ along the longitudinal direction (I). The radius of curvature of the aforementioned curved surfaces 115a may be different. E.g. the radius of curvature of retention unit 110c may be greater than the radius of curvature of retention unit 110a. FIG. 9c show a further example where the radius of curvature of the retention units 110a-d varies with their respective positions along the longitudinal direction (I) of the stent 105. This provides for optimizing the retention force of the retention units 110a-d in dependence on which part on the stent 105 the retention units 110a-d are placed. The stent 105 has a certain extension in the CS and the surrounding anatomy varies along the different parts of the stent 105. The placement and curvatures of the retention units 110a-d may thus be optimized to such varying anatomy to further increase the reliability of the anchoring.

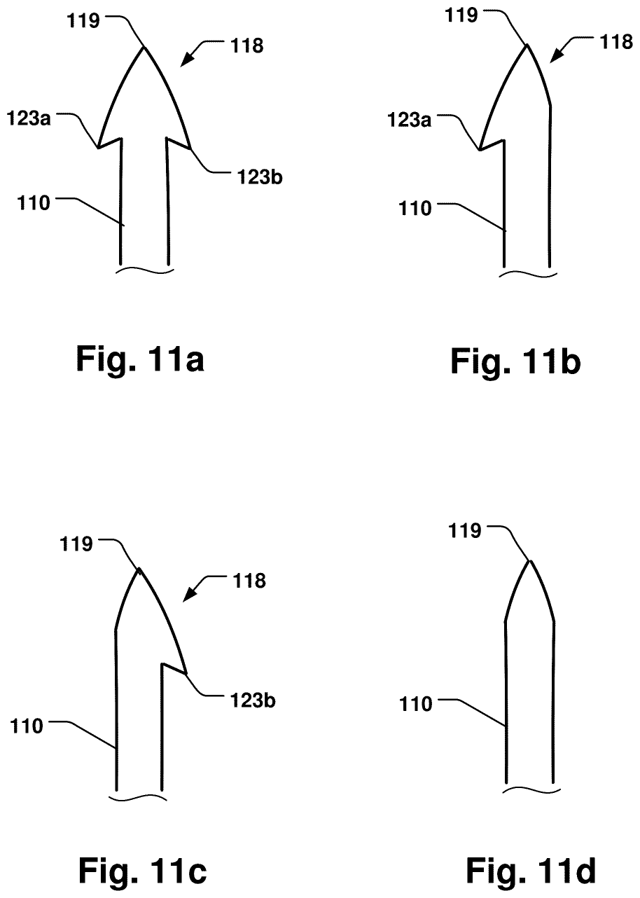

The retention units 110a-d may have a serrated or hook shaped spike 118, as schematically illustrated in FIGS. 10 and 11a-c. This provides for further optimizing the retention force into the tissue. FIG. 11a shows an example where the retention unit 110 tapers to a tip 119 and having an arrow shape with hooks 123a-b at either side of the tip 119. FIG. 11b shows a retention unit 110 with a tip 119 and a single hook 123a at one side of the tip 119. FIG. 11c shows an example where the hook 123b is positioned at the opposite side of the tip 119, compared to FIG. 11b, and FIG. 11d shows a retention unit 110 tapering to a single tip 119 without hooks. Any of the curved retention units 110a-d as described above may have a serrated or hook shaped spike 118. FIG. 10 shows an example where retention units 110a-d have hook-shaped spikes 118. Retention units 110b and 110d are curved whereas retention units 110a and 110c are essentially straight. In one example the hook 123b is provided at the concave surface 115a of the retention unit 110a-d, e.g. as illustrated for retention units 110b and 110d in FIG. 11. This may provide for an increased retention force into the tissue when combined with the curvature of the concave surface 115a.

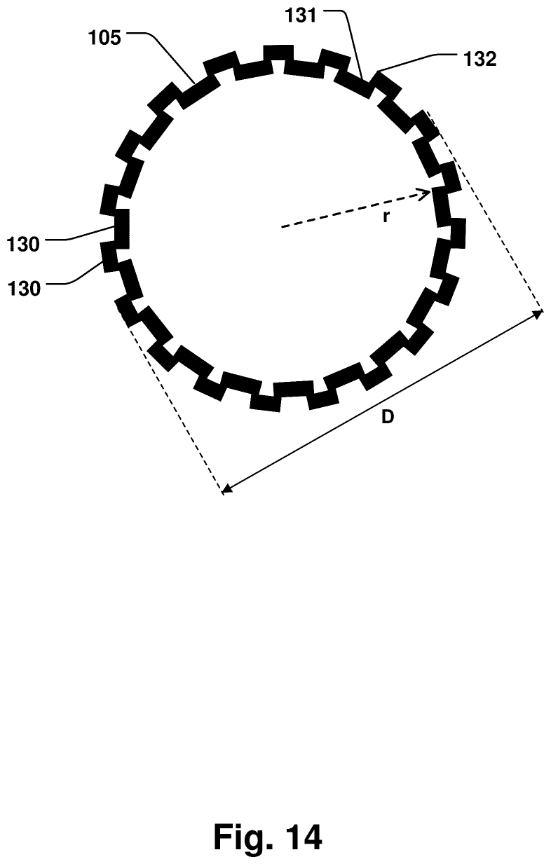

The stent 105 may comprise a framework of a plurality of support elements 130. The support elements 130 are movable in relation to eachother so that the stent 105 is expandable in a radial direction (r), whereby a diameter (D) of the stent 105 is variable. At least part of, or a portion of, the plurality of support elements 130 may be arranged to form an undulating pattern of the stent 105 with recesses 131 and protrusions 132 extending in the radial direction (r). FIG. 14 is a schematic illustration of a cross-section of the stent 105, showing an example of an undulating pattern of the support elements 130 in the cross-sectional view. The support elements 130 thus form recesses 131 and protrusions 132. The protrusions 132 may accordingly engage the tissue of the CS, when the stent 105 is expanded in the CS, as described above, to thereby create a retention force into the tissue. The protrusions 132 thus increase the frictional force between the tissue of the CS and stent 105 for providing anchoring of the stent 105 in the CS. Having the support elements 130 of the framework of the stent 105 forming protrusions 132 for such anchoring provides for a robust anchoring mechanism as well as a facilitated manufacturing of the stent 105 with a minimal amount of separate elements.

The distal anchoring portion 103 may comprise an inflatable unit, such as a balloon, which is expandable in the radial direction (R). This provides for efficient and non-traumatic fixation of the distal end of the displacement unit 101, which in combination with the efficient anchoring against the wall of the CS by the proximal portion 102, allows for an efficient transfer of a contracting force of the proximal and distal portions 102, 103, towards each other. This allows for an effective modification of the radius of curvature of the CS to facilitate modifying the shape of the valve annulus. The annuloplasty device 100 may comprise an inflation lumen (not shown) connected to the inflatable unit and being configured to deliver an inflation medium to the inflatable unit.

The length of inflatable unit 103 may be adapted to varying anatomies. The length of the inflatable unit 103 may be chosen so that it does not block vessels connecting to the CS, e.g. if the inflatable unit 103 is anchored further into the CS, such as towards the great cardiac vein/left coronary vein. The length of the inflatable unit 103 may also be adapted so that it may be effectively anchored behind the bend or “corner” of the CS as it transitions into the great cardiac vein/left coronary vein. The length of the inflatable unit 103 may be sufficiently short to facilitate such anchoring and avoid slipping out of this bend or “corner” of the CS.

The proximal expandable portion 102 may comprise expandable bows or ribs 112. The sheath 107 may be pushed in relation to a distal portion 114 attached distally to the bows or ribs 112. The compressive force between the distal portion 114 and the proximal portion 108 may thus push the bows 112 radially outwards. It is conceivable however that the bows 112 may comprise a shape-memory material having a tendency to assume the expanded configuration in its relaxed state, and that the bows may be confined in an outer sheath (not shown) being pulled back so that the bows 112 spring into the expanded configuration.

Having expandable bows 112 provides for to further lessen the risk of damaging the tissue at the entrance of the CS, since a soft apposition against the tissue may be provided, in absence of sharp edges or kinks. The bows 112 may extend in the longitudinal direction 104 which facilitates a symmetric engagement against the tissue wall, with an even transfer of force around the entrance to the CS, hence allowing for a robust anchoring. The longitudinal extension of the bows 112 also provides for facilitated expansion of the bows 112 by applying a force to the bows 112 in the longitudinal direction 104. A plurality of bows 112 may be arranged circumferentially so that a force may be applied symmetrically and evenly around the tissue wall.

The proximal expandable portion 102 may comprise elongated ribs 112 formed in the sheath 107 by elongated cuts 113 in the sheath 107, extending in the longitudinal direction 104, as schematically illustrated in FIG. 2a. The ribs 112 may be foldable to expand in the radial direction (R). This provides for a simple and robust construction. The ribs or bows 112 may thus be formed from the same material as the sheath 107. The mentioned material may be a soft flexible material which is non-traumatic to tissue. In the collapsed configuration seen in FIG. 2a, the ribs 112, i.e. the soon to be expanded ribs 112, extend in the longitudinal direction 104, and provides for a compact radial profile. The ribs or bows 112 may be placed equidistantly around a circumference of the sheath 107. As elucidated above, this may provide for an even distribution of the anchoring force.

The maximum expanded diameter (D) of the proximal expandable portion 102 may be at least three times the diameter of the CS. In some examples the ratio of the maximum expanded diameter (D) of the proximal expandable portion 102 to the diameter of the CS is in the range 3-5. In some examples the aforementioned ratio may be in the range 3.5-4.5, which provides for a particular advantageous anchoring of the proximal expandable portion 102, while maintaining a compact and easy to use annuloplasty device 100.

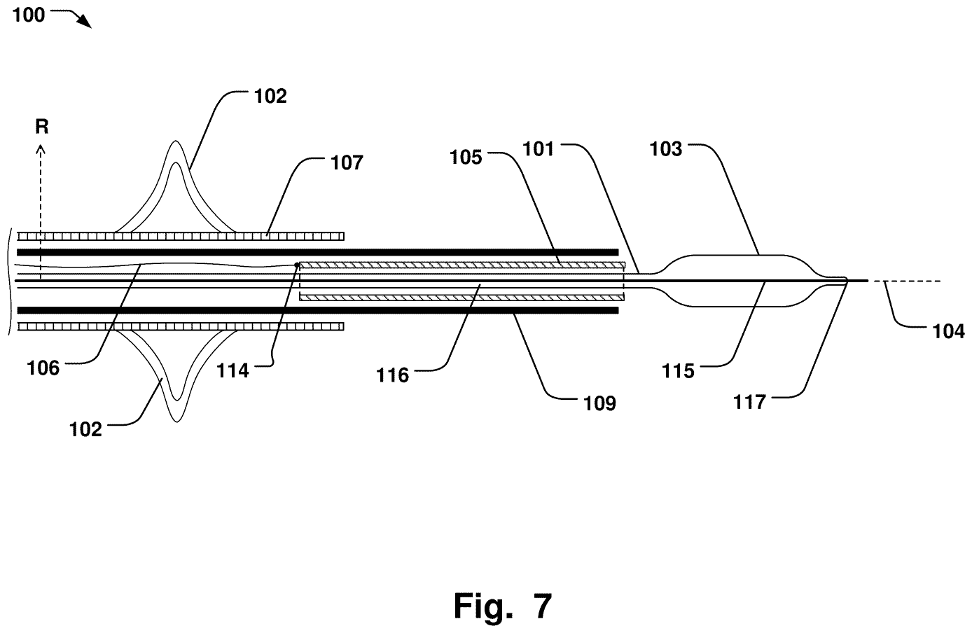

The annuloplasty device 100 may comprise a guide wire 115 arranged to extend inside a lumen 116 of the displacement unit 101 and to exit the lumen 116 at a distal opening 117 of the displacement unit 101, as schematically illustrated in FIG. 7. The guide wire 115 may be inserted into the CS and the displacement unit 101 may subsequently be advanced over the guide wire 115 for positioning in the CS. This provides for a facilitated positioning of the displacement unit 101.

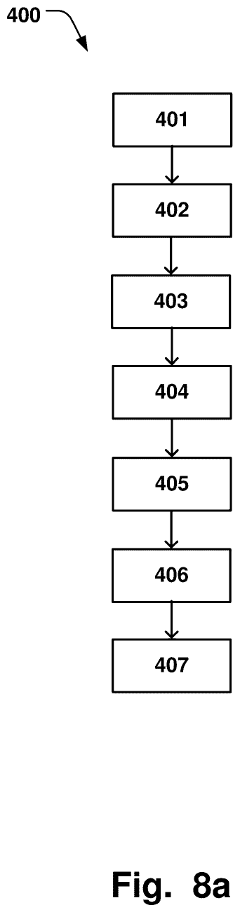

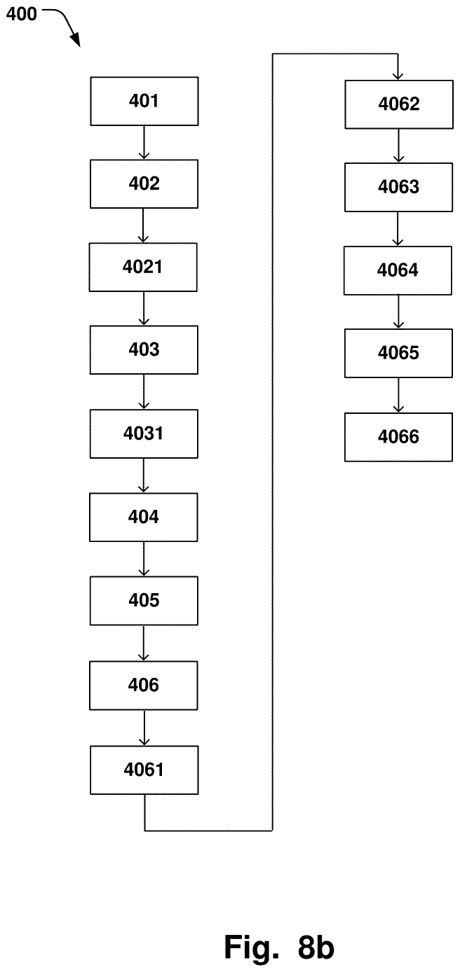

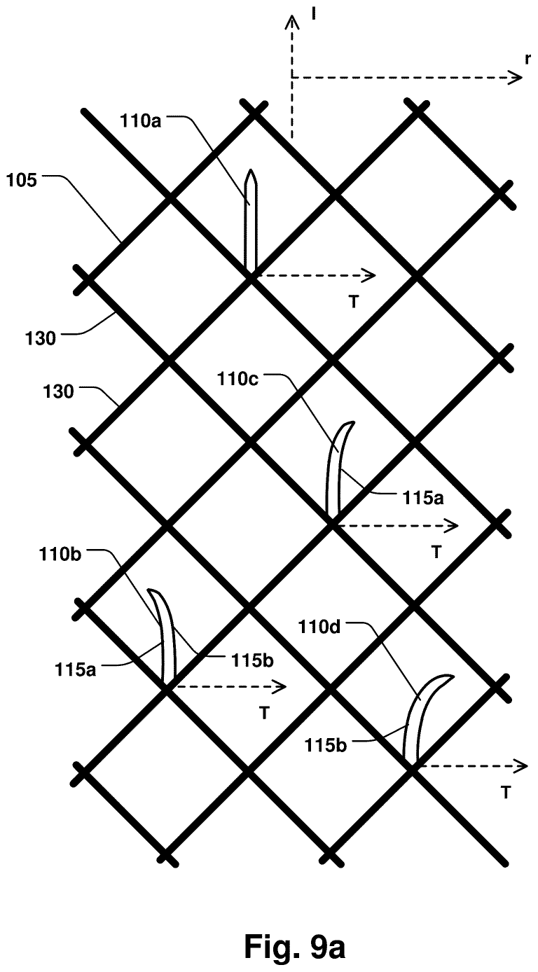

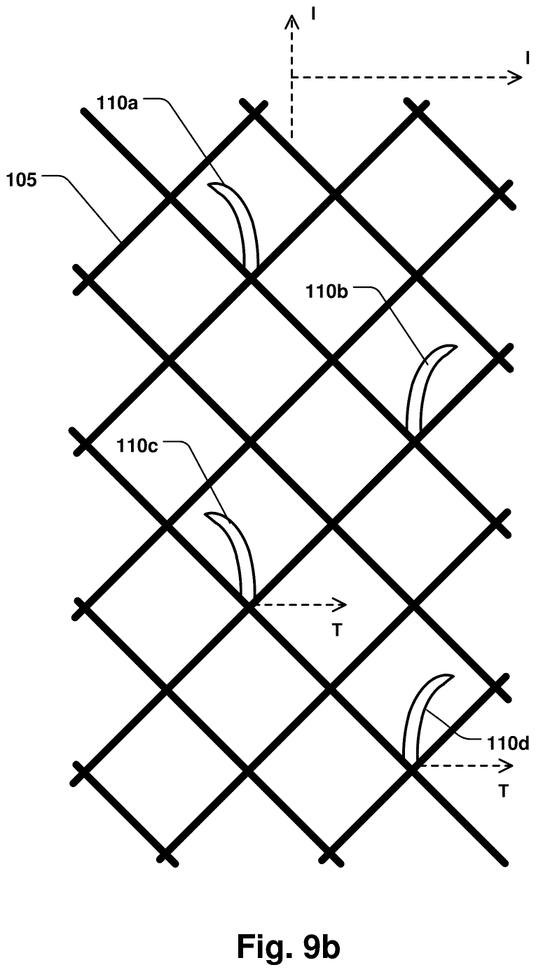

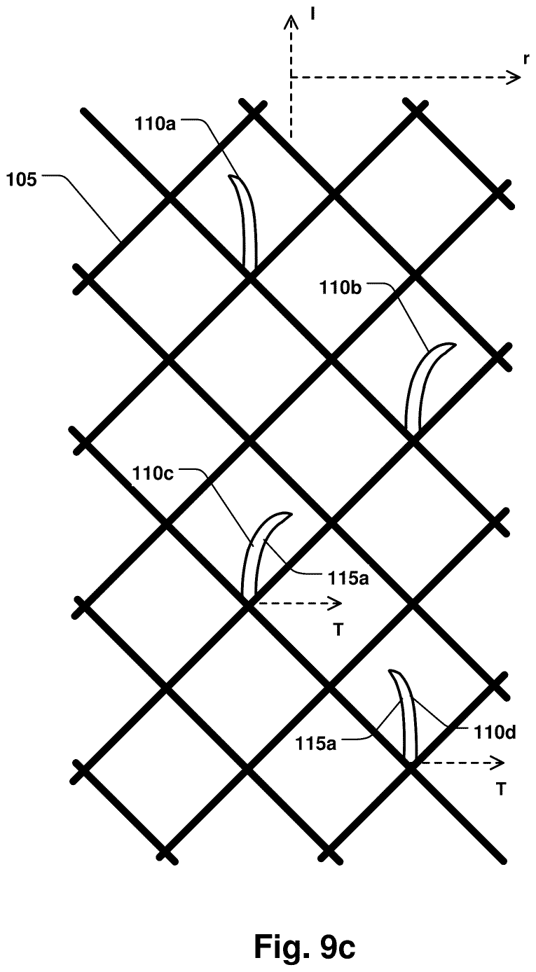

FIG. 8a illustrates a method 400 for treating a defective mitral valve. The order in which the steps of the method 400 are illustrated should not be construed as limiting and it is conceivable that the order in which the steps of the method 400 is carried out may be varied. The method 400 comprises inserting 401 a flexible and removable elongate displacement unit 101 in a delivery state into a coronary sinus CS adjacent said valve; positioning 402 a proximal reversibly expandable portion 102 against a tissue wall at the entrance of said CS (FIG. 5a); anchoring 403 a distal anchoring portion 103 inside the CS (FIG. 5b); activating 404 the displacement unit 101 in an activated state whereby the distal anchoring portion 103 is moved in a longitudinal direction 104 of the displacement unit 101 to reduce a distance (L) between the distal anchoring portion 103 and the proximal expandable portion 102 such that the shape of the annulus is modified to a modified shape. The method 400 further comprises advancing 405 a stent 105 through the proximal expandable portion 102 and over the displacement unit 101 into the CS (FIGS. 5e-f); anchoring 406 the stent 105 in the CS to retain the modified shape of the annulus (FIGS. 5g-i); withdrawing 407 the displacement unit 101 through the stent 105 to remove 408 the displacement unit 101 after temporary activation in the activated state (FIGS. 5j-l, 6b). The method 400 thus provides for the advantageous benefits as described above in relation to the annuloplasty device 100 and FIGS. 1-7. The method 400 provides for an improved annuloplasty procedure with an increase in the degree of control of the downsizing procedure, while ensuring secure anchoring of the stent 105 and minimal risk of damage to the CS. Advancing the stent 105 over the displacement unit 101 into the CS while the proximal expandable portion 102 is expanded at the outside of the CS and the displacement unit 101 is in the activated state provides for a secure positioning and fixation of the stent 105 when the valve is already re-shaped by the displacement unit 101. A particularly robust and reliable annuloplasty procedure is thus provided.

FIG. 6b illustrates another flow chart of a method 400 for treating a defective mitral valve. The order in which the steps of the method 400 are illustrated should not be construed as limiting and it is conceivable that the order in which the steps of the method 400 is carried out may be varied.

The distal anchoring portion 103 may comprise an inflatable unit, also denoted with reference numeral 103. Anchoring the distal anchoring portion 103 may comprise inflating 4031 the inflatable unit 103 in the coronary sinus, and/or in the great cardiac vein and/or, in the anterior interventricular branch or vein, and/or in the posterior vein and/or in the posterior ventricular vein of the heart.

The proximal expandable portion 102 may be connected to a sheath 107. Positioning the proximal expandable portion 102 may comprise pushing 4021 a proximal portion 108 of the sheath 107 towards the distal anchoring portion 103 to expand the proximal expandable portion 102 in a radial direction (R).

Anchoring the stent 105 may comprise withdrawing 4061 a catheter 109 enclosing the stent 105 and expanding 4062 the stent 105 in a radial direction (R) being perpendicular to the longitudinal direction 104, as schematically illustrated in FIGS. 5g-h.

The catheter 109 may be is movable through the proximal expandable portion 102 and over the displacement unit 101 in the longitudinal direction 104, providing for the advantageous effects as described above.

Anchoring the stent 105 may comprise anchoring 4063 retention units 110 of the stent 105 into the CS to retain the modified shape of the annulus when the displacement unit 101 is withdrawn, as schematically illustrated in FIGS. 5g-l.

Anchoring the retention units 110 may comprise anchoring 4064 the retention units 110 in a tissue wall of the CS in the direction of the annulus.

The method 400 may comprise advancing 4065 the catheter 109 over the stent 105 to disengage the stent 105 from the CS for repositioning or removal of the stent 105 from the CS.

Anchoring the stent 105 may comprise releasing 4066 the stent 105 from a delivery device 106 movably arranged inside the catheter 109 (FIG. 5k).

The present invention has been described above with reference to specific embodiments. However, other embodiments than the above described are equally possible within the scope of the invention. The different features and steps of the invention may be combined in other combinations than those described. The scope of the invention is only limited by the appended patent claims.

More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

1-32. (canceled)

33. An annuloplasty device for treating a defective mitral valve having an annulus, said annuloplasty device comprising:
a removable and flexible elongate displacement unit for temporary insertion into a coronary sinus (CS) adjacent the valve, wherein the displacement unit has a delivery state for delivery into the CS, and an activated state to which the displacement unit is temporarily and reversibly transferable from said delivery state, a proximal reversibly expandable portion being reversibly foldable to an expanded state for positioning against a tissue wall at the entrance of the CS,
wherein the displacement unit comprises a distal anchoring portion being movable in relation to the proximal expandable portion in a longitudinal direction of the displacement unit to said activated state in which the shape of the annulus is modified to a modified shape when inserted in the CS, and
a stent arranged around the displacement unit and being movable relative the displacement unit along the longitudinal direction for insertion into the CS,
wherein the stent is releasably connected to a delivery device and arranged radially between the displacement unit and the proximal expandable portion in a radial direction (R), the radial direction (R) being perpendicular to the longitudinal direction, and wherein the stent comprises retention units to anchor the stent in the CS.

34. The annuloplasty device according to claim 33, wherein the proximal expandable portion is connected to a sheath and is configured to be expanded in the radial direction (R) by pushing a proximal portion of the sheath towards the distal anchoring portion.

35. The annuloplasty device according to claim 33, further comprising a catheter to enclose the stent and position the stent relative the displacement unit in the longitudinal direction, wherein the stent is ejectable from, and retrievable into, the catheter by the delivery device, wherein the catheter is movable over the displacement unit, and inside said sheath, in the longitudinal direction, in said activated state.

36. The annuloplasty device according to claim 33, wherein the retention units are arranged on a defined surface section of the stent adapted to be arranged towards the annulus when the stent is in the CS.

37. The annuloplasty device according to claim 33, wherein a plurality of retention units are arranged around a circumference of the stent.

38. The annuloplasty device according to claim 35, wherein the retention units are resiliently moveable from a retracted state to an expanded state, whereby the retention units are flexible to bend from the expanded state to the retracted state when arranged inside the catheter and expand from the retracted state to the expanded state when released from the catheter.

39. The annuloplasty device according to claim 38, wherein the retention units are aligned essentially flush with an outer diameter of the stent in the retracted state.

40. The annuloplasty device according to claim 33, wherein the proximal expandable portion comprise elongated ribs formed in a sheath by elongated cuts in the sheath, extending in the longitudinal direction, the ribs being foldable to expand in the radial direction (R).

41. The annuloplasty device according to claim 33, wherein the retention units have a curved shape comprising an at least partly concave or convex surface towards the direction of a respective tangent vector (T) at the respective positions of the retention units on the stent, the tangent vector is perpendicular to a radial direction (r) of the stent and a longitudinal direction (I) of the stent, and the radial direction is perpendicular to the longitudinal direction.

42. The annuloplasty device according to claim 41, wherein the retention units are curved in opposite directions so that a convex surface of at least a first retention unit faces the opposite direction of a convex surface of at least a second retention unit.

43. The annuloplasty device according to claim 42, wherein the first retention unit is curved towards the second retention unit.

44. The annuloplasty device according to claim 42, wherein the first and second retention units are curved away from each other.

45. The annuloplasty device according to claim 41, wherein the amount of curvature of the curved shape varies with an angle (v1, v2) at which the respective retention units are positioned around a circumference of the stent.

46. The annuloplasty device according to claim 41, wherein the amount of curvature of the curved shape varies with a position (d1, d2) along the longitudinal direction (L) of the stent at which the respective retention unit is positioned.

47. The annuloplasty device according to claim 41, wherein the retention units have a serrated or hook shaped spike.

48. The annuloplasty device according to claim 33, wherein the stent comprises a framework of a plurality of support elements, wherein the support elements are movable in relation to each other so that the stent is expandable in a radial direction (r), whereby a diameter (D) of the stent is variable, wherein at least part of the plurality of support elements are arranged to form an undulating pattern of the stent with recesses and protrusions extending in the radial direction.

49. A method for treating a defective mitral valve having an annulus, said method comprising:
inserting a flexible and removable elongate displacement unit in a delivery state into a coronary sinus (CS) adjacent said valve,
positioning a proximal reversibly expandable portion against a tissue wall at the entrance of said CS,
anchoring a distal anchoring portion inside the CS,
activating the displacement unit in an activated state whereby the distal anchoring portion is moved in a longitudinal direction of the displacement unit to reduce a distance (L) between the distal anchoring portion and the proximal expandable portion such that the shape of the annulus is modified to a modified shape,
advancing a stent through the proximal expandable portion and over the displacement unit into the CS,
anchoring the stent in the CS to retain the modified shape of the annulus, and
withdrawing the displacement unit through the stent to remove the displacement unit after temporary activation in the activated state.

50. The method according to claim 49, wherein the distal anchoring portion comprises an inflatable unit and anchoring the distal anchoring portion comprises inflating the inflatable unit in the coronary sinus, and/or in the great cardiac vein and/or, and/or in the anterior interventricular branch or vein, and/or in the posterior vein, and/or in the posterior ventricular vein of the heart.

51. The method according to claim 49, wherein the proximal expandable portion is connected to a sheath, and said positioning the proximal expandable portion comprises pushing a proximal portion of the sheath towards the distal anchoring portion to expand the proximal expandable portion in a radial direction (R).

52. The method according to claim 49, wherein said anchoring the stent comprises withdrawing a catheter enclosing the stent and expanding the stent in a radial direction (R) being perpendicular to the longitudinal direction.

53. The method according to claim 52, wherein the catheter is movable through the proximal expandable portion and over the displacement unit in the longitudinal direction.

54. The method according to claim 52, wherein said anchoring the stent comprises anchoring retention units of the stent into the CS to retain the modified shape of the annulus when the displacement unit is withdrawn.

55. The method according to claim 54, wherein said anchoring the retention units comprises anchoring the retention units in a tissue wall of the CS in the direction of the annulus.

56. The method according to claim 52, further comprising advancing the catheter over the stent to disengage the stent from the CS for repositioning or removal of the stent from the CS.

57. The method according to claim 52, wherein said anchoring the stent comprises releasing the stent from a delivery device movably arranged inside the catheter.
